(19) United States
(12) Patent Application Publication
(10) Pub. No.: US 2006/0222886 A1
(43) Pub. Date: Oct. 5, 2006

(54) Arylpyrene compounds

(76) Inventors: Raymond Kwong, Plainsboro, NJ (US); Matthew Nugent, Levittown, PA (US)

Correspondence Address: Kenyon & Kenyon, Suite 700, 1500 K Street, Washington, DC, 20005, US

(21) Appl. No.: 11/097,352

(22) Filed: Apr. 4, 2005

(51) Int. Cl. H01L 51/54; C07C 15/20; H05B 33/14; C09K 11/06; C07C 15/56

(52) U.S. Cl. 428/690; 585/26; 428/917; 313/504; 257/E51.049; 585/27

(57) ABSTRACT

One embodiment of the present invention provides an organic light emitting device comprising an anode, a cathode, and an organic layer disposed between the anode and the cathode, wherein the organic layer comprises an arylpyrene compound. In another embodiment, the present invention also provides organic light emitting devices comprising an anode, a cathode, and an organic layer disposed between the anode and the cathode, wherein the organic layer comprises an arylpyrene compound doped in an anthracene host, preferably ADN. Another embodiment of the present invention provides specific arylpyrene compounds.

### FIELD OF THE INVENTION

The present invention relates to organic light emitting devices (OLEDs), and more specifically to OLEDs with an emissive region comprising an arylpyrene compound.

### BACKGROUND

Opto-electronic devices that make use of organic materials are becoming increasingly desirable for a number of reasons. Many of the materials used to make such devices are relatively inexpensive, so organic opto-electronic devices have the potential for cost advantages over inorganic devices. In addition, the inherent properties of organic materials, such as their flexibility, may make them well suited for particular applications such as fabrication on a flexible substrate. Examples of organic opto-electronic devices include organic light emitting devices (OLEDs), organic phototransistors, organic photovoltaic cells, and organic photodetectors. For OLEDs, the organic materials may have performance advantages over conventional materials. For example, the wavelength at which an organic emissive layer emits light may generally be readily tuned with appropriate dopants.

As used herein, the term “organic” includes polymeric materials as well as small molecule organic materials that may be used to fabricate organic opto-electronic devices. “Small molecule” refers to any organic material that is not a polymer, and “small molecules” may actually be quite large. Small molecules may include repeat units in some circumstances. For example, using a long chain alkyl group as a substituent does not remove a molecule from the “small molecule” class. Small molecules may also be incorporated into polymers, for example as a pendent group on a polymer backbone or as a part of the backbone. Small molecules may also serve as the core moiety of a dendrimer, which consists of a series of chemical shells built on the core moiety. The core moiety of a dendrimer may be a fluorescent or phosphorescent small molecule emitter. A dendrimer may be a “small molecule,” and it is believed that all dendrimers currently used in the field of OLEDs are small molecules. In general, a small molecule has a well-defined chemical formula with a single molecular weight, whereas a polymer has a chemical formula and a molecular weight that may vary from molecule to molecule. As used herein, “organic” includes metal complexes of hydrocarbyl and heteroatom-substituted hydrocarbyl ligands.

OLEDs make use of thin organic films that emit light when voltage is applied across the device. OLEDs are becoming an increasingly interesting technology for use in applications such as flat panel displays, illumination, and backlighting. Several OLED materials and configurations are described in U.S. Pat. Nos. 5,844,363, 6,303,238, and 5,707,745, which are incorporated herein by reference in their entirety.

OLED devices are generally (but not always) intended to emit light through at least one of the electrodes, and one or more transparent electrodes may be useful in an organic opto-electronic devices. For example, a transparent electrode material, such as indium tin oxide (ITO), may be used as the bottom electrode. A transparent top electrode, such as disclosed in U.S. Pat. Nos. 5,703,436 and 5,707,745, which are incorporated by reference in their entireties, may also be used. For a device intended to emit light only through the bottom electrode, the top electrode does not need to be transparent, and may be comprised of a thick and reflective metal layer having a high electrical conductivity. Similarly, for a device intended to emit light only through the top electrode, the bottom electrode may be opaque and/or reflective. Where an electrode does not need to be transparent, using a thicker layer may provide better conductivity, and using a reflective electrode may increase the amount of light emitted through the other electrode, by reflecting light back towards the transparent electrode. Fully transparent devices may also be fabricated, where both electrodes are transparent. Side emitting OLEDs may also be fabricated, and one or both electrodes may be opaque or reflective in such devices.

As used herein, “top” means furthest away from the substrate, while “bottom” means closest to the substrate. For example, for a device having two electrodes, the bottom electrode is the electrode closest to the substrate, and is generally the first electrode fabricated. The bottom electrode has two surfaces, a bottom surface closest to the substrate, and a top surface further away from the substrate. Where a first layer is described as “disposed over” a second layer, the first layer is disposed further away from substrate. There may be other layers between the first and second layer, unless it is specified that the first layer is “in physical contact with” the second layer. For example, a cathode may be described as “disposed over” an anode, even though there are various organic layers in between.

As used herein, “solution processible” means capable of being dissolved, dispersed, or transported in and/or deposited from a liquid medium, either in solution or suspension form.

As used herein, and as would be generally understood by one skilled in the art, a first “Highest Occupied Molecular Orbital” (HOMO) or “Lowest Unoccupied Molecular Orbital” (LUMO) energy level is “greater than” or “higher than” a second HOMO or LUMO energy level if the first energy level is closer to the vacuum energy level. Since ionization potentials (IP) are measured as a negative energy relative to a vacuum level, a higher HOMO energy level corresponds to an IP having a smaller absolute value (an IP that is less negative). Similarly, a higher LUMO energy level corresponds to an electron affinity (EA) having a smaller absolute value (an EA that is less negative). On a conventional energy level diagram, with the vacuum level at the top, the LUMO energy level of a material is higher than the HOMO energy level of the same material. A “higher” HOMO or LUMO energy level appears closer to the top of such a diagram than a “lower” HOMO or LUMO energy level.

### SUMMARY OF THE INVENTION

One embodiment of the present invention provides an organic light emitting device comprising an anode, a cathode, and an organic layer disposed between the anode and the cathode, wherein the organic layer comprises an arylpyrene compound of formula I: 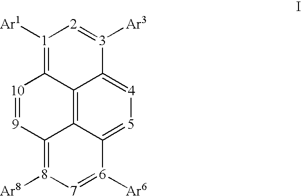
wherein each of Ar¹, Ar³, Ar⁶, and Ar⁸ is independently a 2-napthyl group of structure II: 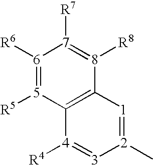
wherein each of R⁴⁻⁸ is an independently selected substituent, and wherein each of Ar¹, Ar³, Ar⁶, and Ar⁸ has a hydrogen at positions 1 and 3.

In one embodiment, the arylpyrene compound is a compound of formula III:

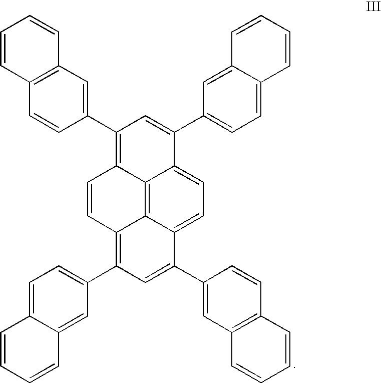

In one embodiment, the present invention also provides these arylpyrene compounds per se.

In one embodiment, the arylpyrene compound has an angle defined by the plane of the pyrene core and the plane of the Ar¹, Ar³, Ar⁶, or Ar⁸ group that is less than about 60 degrees.

In one embodiment, the arylpyrene compound is doped in a host such as an anthracene host, preferably ADN, or a carbazole host, preferably CBP. In another embodiment, the arylpyrene compound is deposited as a neat layer.

In one embodiment, the arylpyrene compound has a peak in the emission spectra that is less than about 500 nm. In another embodiment, the arylpyrene compound emits light with CIE coordinates of (X≦0.2, Y≦0.3).

In yet another embodiment, the device of the present invention has an unmodified external quantum efficiency is greater than about 5%.

### BRIEF DESCRIPTION OF THE DRAWINGS

FIG. 1 shows an organic light emitting device having separate electron transport, hole transport, and emissive layers, as well as other layers.

FIG. 2 shows an inverted organic light emitting device that does not have a separate electron transport layer.

FIG. 3 shows the PL and CIE of neat 1,3,6,8-tetra(2-naphthyl)pyrene.

FIG. 4 shows plots comparing current density (mA/cm²) vs. voltage (V) for Examples 4 and 5.

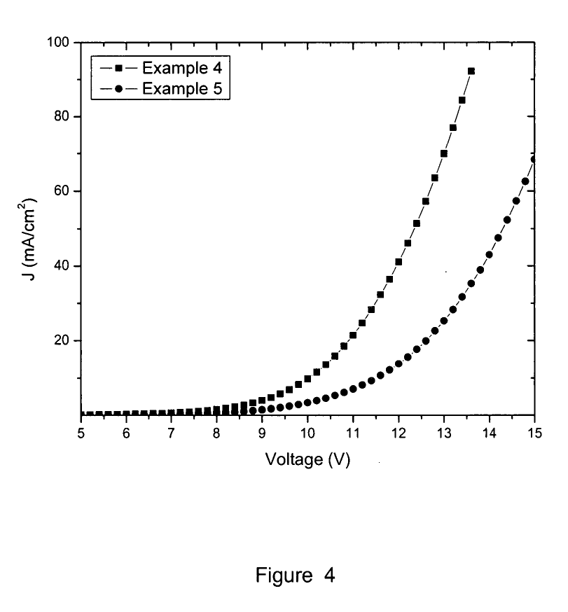

FIG. 5 shows plots comparing luminous efficiency (cd/A) vs. brightness (cd/m²) for Examples 4 and 5.

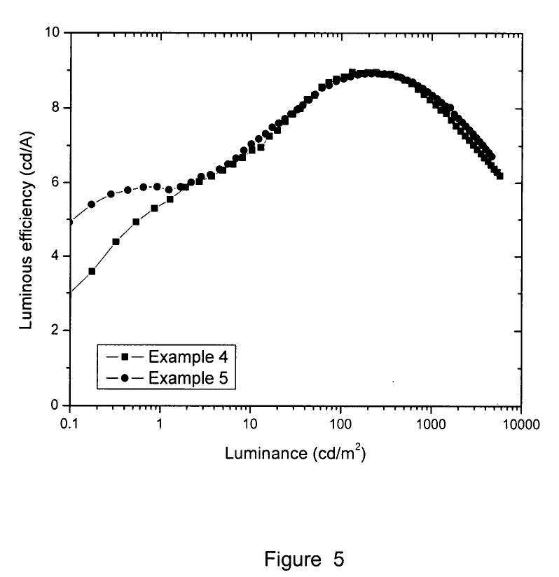

FIG. 6 shows the quantum efficiency (QE) as a function of current density (mA/cm²) for Examples 4 and 5.

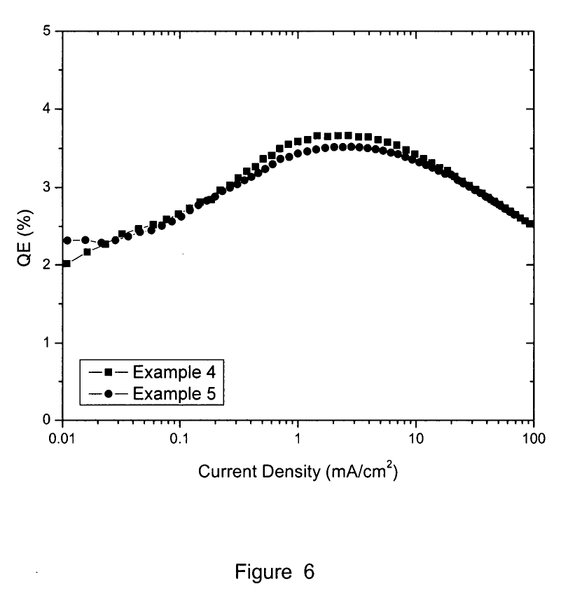

FIG. 7 shows normalized EL intensity versus wavelength (nm) for Examples 4 and 5.

FIG. 8 shows plots comparing current density (mA/cm²) vs. voltage (V) for Examples 6 and 7.

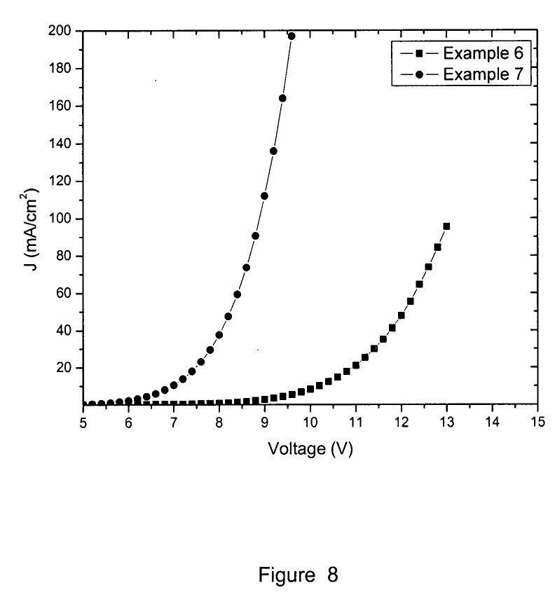

FIG. 9 shows plots comparing luminous efficiency (cd/A) vs. brightness (cd/m²) for Examples 6 and 7.

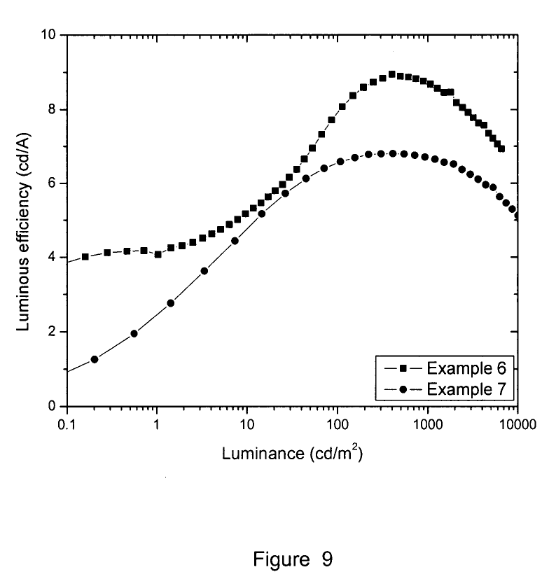

FIG. 10 shows the quantum efficiency (QE) as a function of current density (mA/cm²) for Examples 6 and 7.

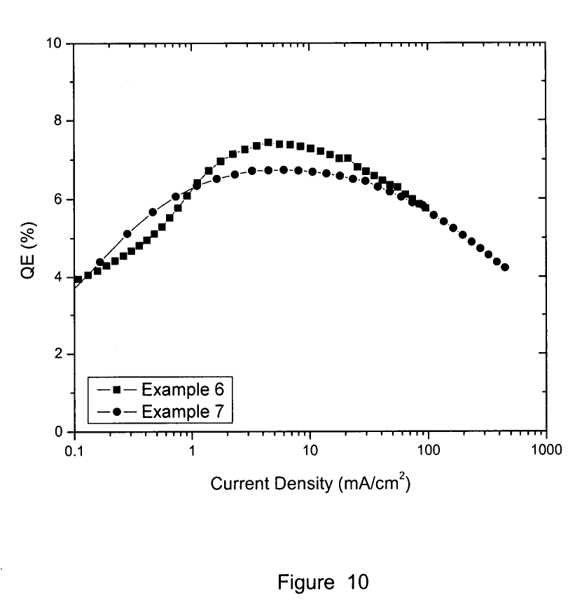

FIG. 11 shows radiance (watts/sr/m²) versus wavelength (nm) for Example 6.

FIG. 12 shows radiance (watts/sr/m²) versus wavelength (nm) for Example 7.

FIG. 13 shows plots comparing current density (mA/cm²) vs. voltage (V) for Examples 8-10.

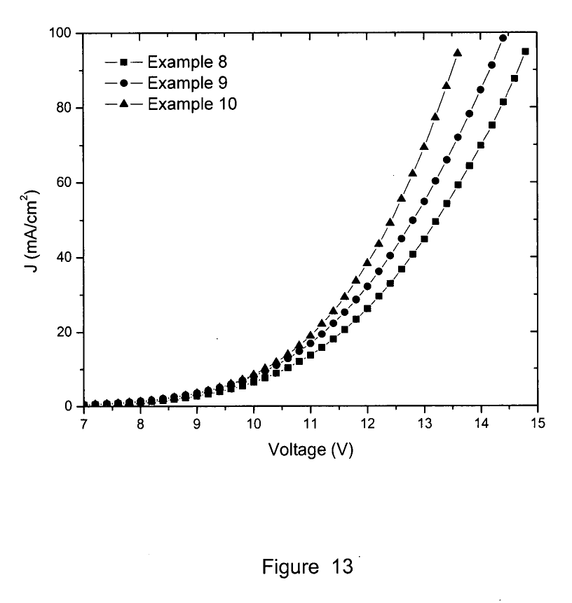

FIG. 14 shows plots comparing luminous efficiency (cd/A) vs. brightness (cd/m²) for Examples 8-10.

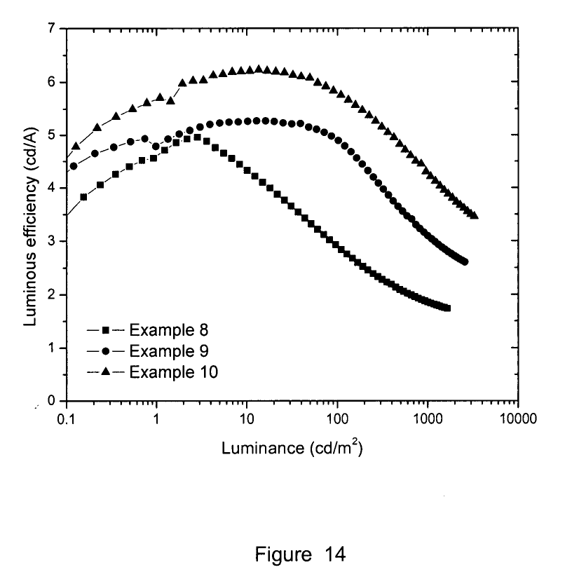

FIG. 15 shows the quantum efficiency (QE) as a function of current density (mA/cm²) for Examples 8-10.

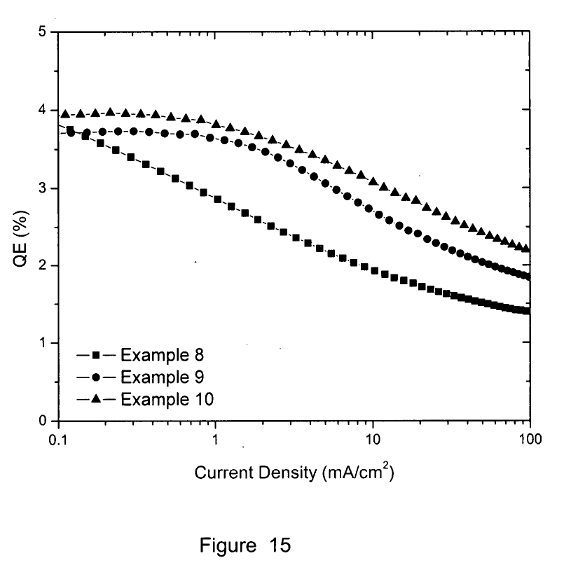

FIG. 16 shows normalized EL intensity versus wavelength (nm) for Examples 8-10.

FIG. 17 shows plots comparing current density (mA/cm²) vs. voltage (V) for Examples 11 and 12.

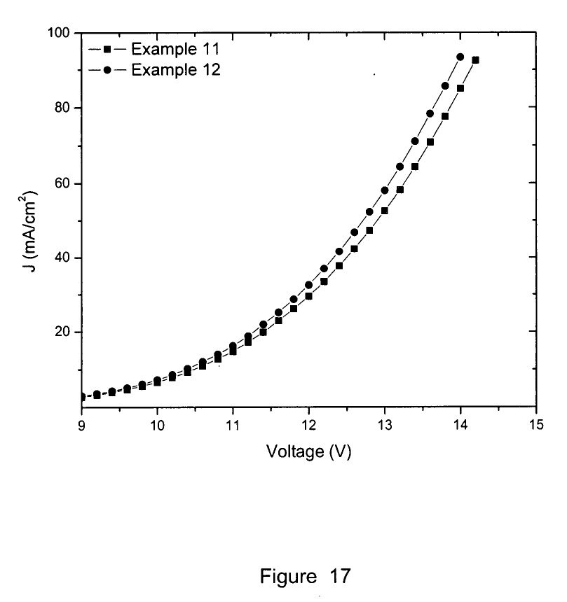

FIG. 18 shows plots comparing luminous efficiency (cd/A) vs. brightness (cd/m²) for Examples 11 and 12.

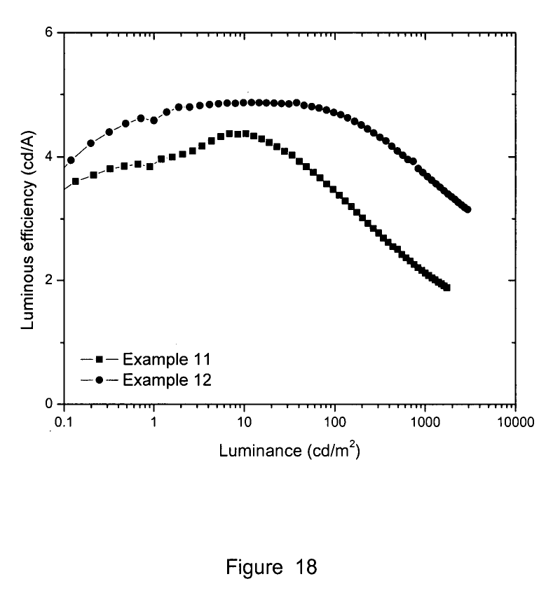

FIG. 19 shows the quantum efficiency (QE) as a function of current density (mA/cm²) for Examples 11 and 12.

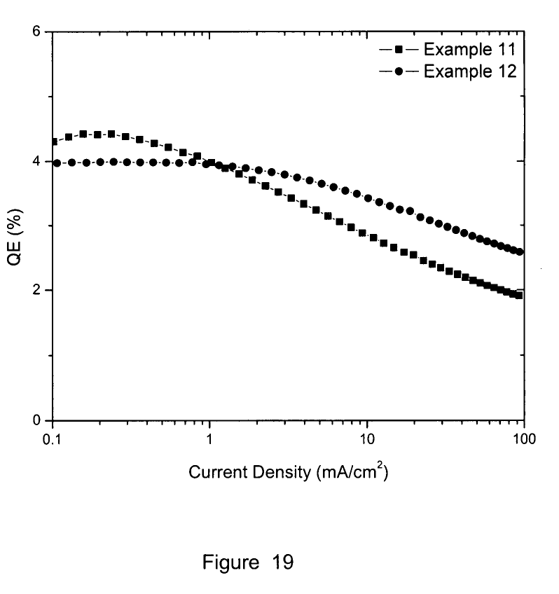

FIG. 20 shows normalized EL intensity versus wavelength (nm) for Examples 11 and 12.

FIG. 21 shows plots comparing current density (mA/cm²) vs. voltage (V) for Examples 13 and 14.

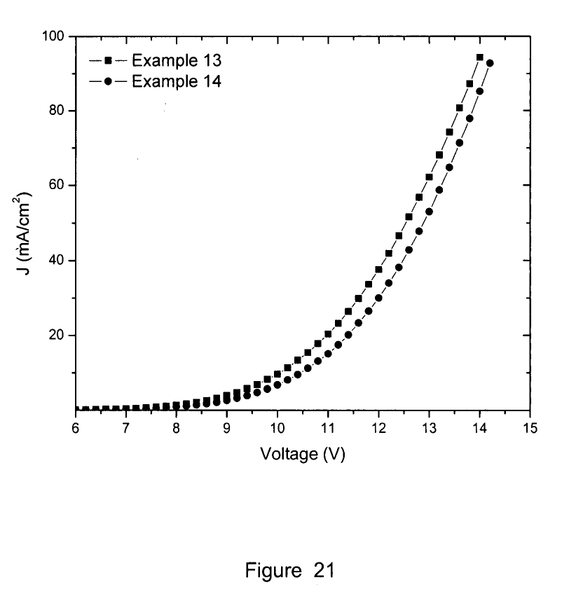

FIG. 22 shows plots comparing luminous efficiency (cd/A) vs. brightness (cd/m²) for Examples 13 and 14.

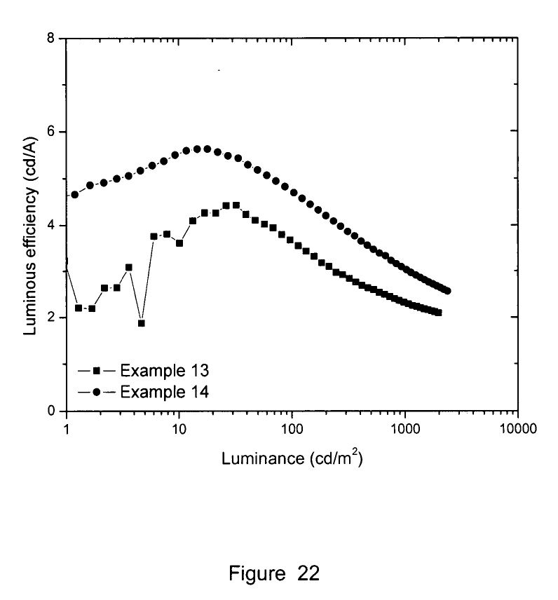

FIG. 23 shows the quantum efficiency (QE) as a function of current density (mA/cm²) for Examples 13 and 14.

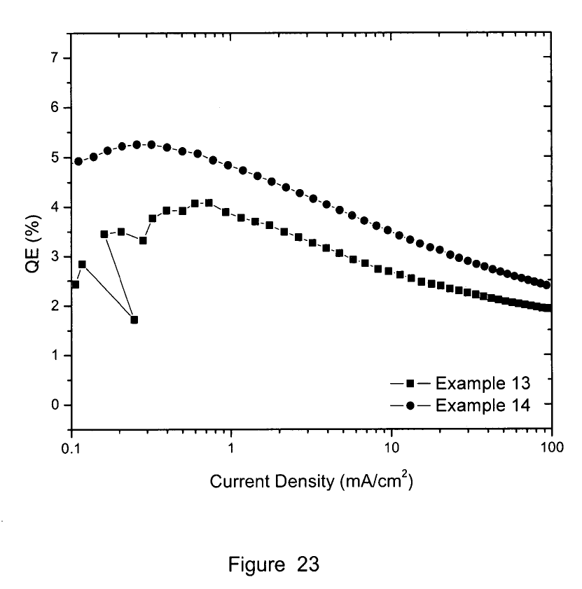

FIG. 24 shows normalized EL intensity versus wavelength (nm) for Examples 13 and 14.

FIG. 25 shows plots comparing current density (mA/cm²) vs. voltage (V) for Examples 15-18.

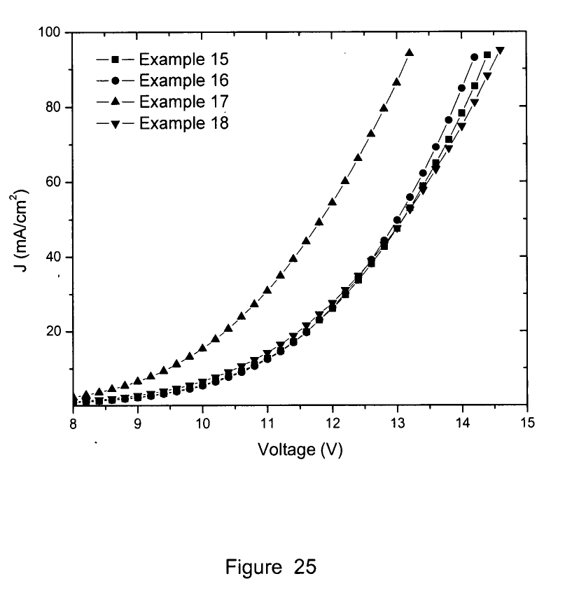

FIG. 26 shows plots comparing luminous efficiency (cd/A) vs. brightness (cd/m²) for Examples 15-18.

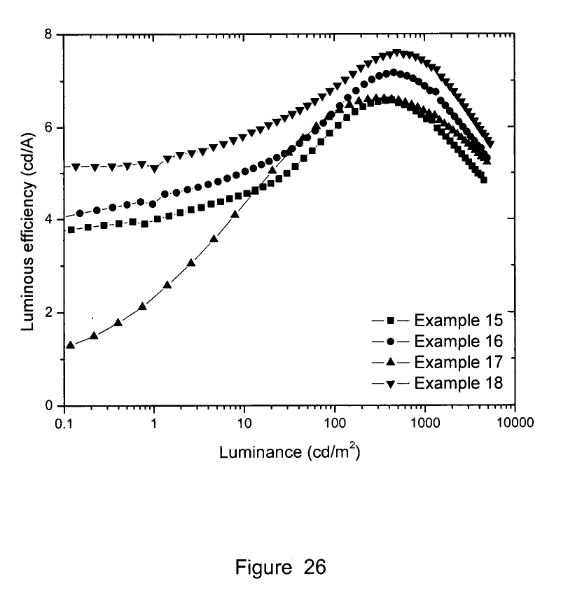

FIG. 27 shows the quantum efficiency (QE) as a function of current density (mA/cm²) for Examples 15-18.

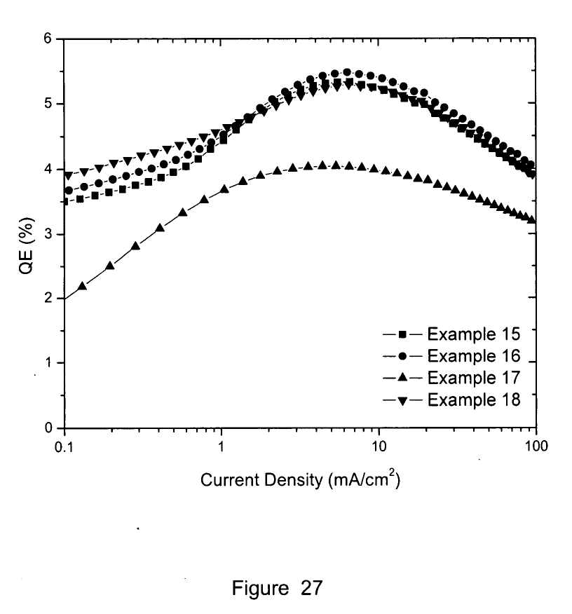

FIG. 28 shows normalized EL intensity versus wavelength (nm) for Examples 15-18.

FIG. 29 shows plots comparing current density (mA/cm²) vs. voltage (V) for Examples 19-23.

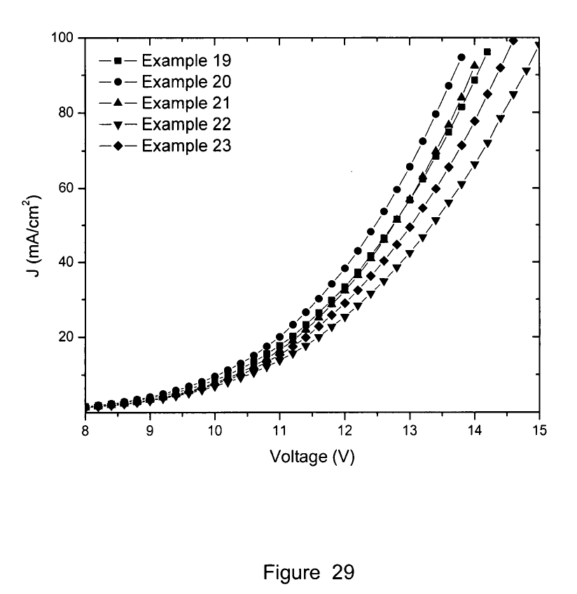

FIG. 30 shows plots comparing luminous efficiency (cd/A) vs. brightness (cd/m²) for Examples 19-23.

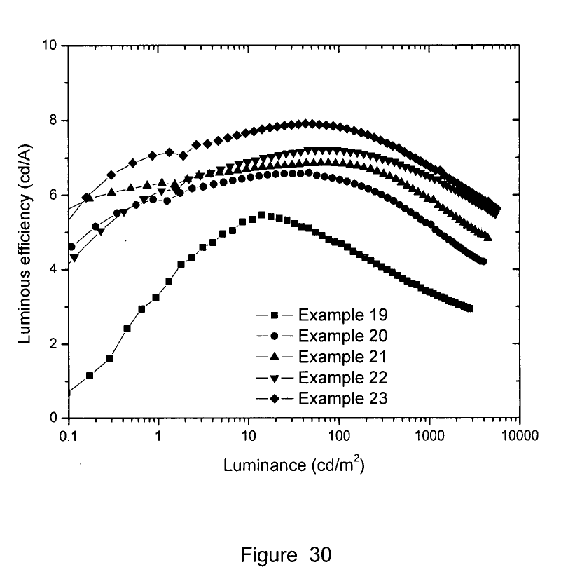

FIG. 31 shows the quantum efficiency (QE) as a function of current density (mA/cm²) for Examples 19-23.

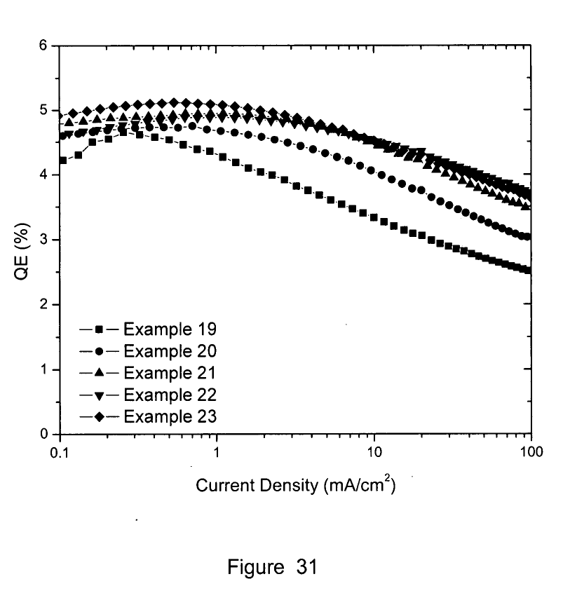

FIG. 32 shows normalized EL intensity versus wavelength (nm) for Examples 19-23.

FIG. 33 shows the operational stability represented by the normalized EL intensity versus operation time for Examples 6 and 7 under a DC current of 5 mA/cm² at room temperature.

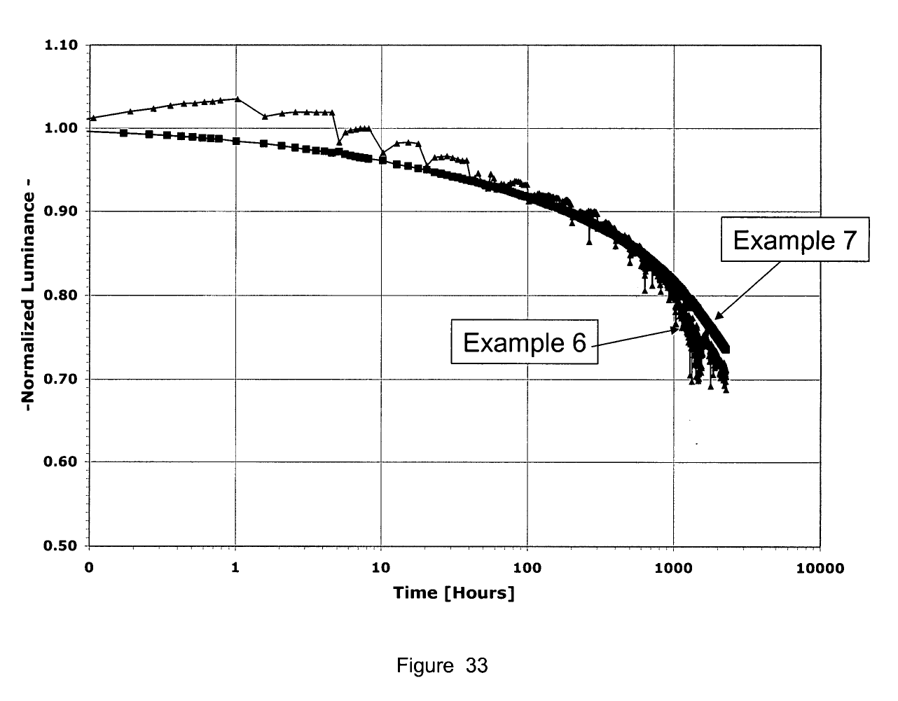

### DETAILED DESCRIPTION

Generally, an OLED comprises at least one organic layer disposed between and electrically connected to an anode and a cathode. When a current is applied, the anode injects holes and the cathode injects electrons into the organic layer(s). The injected holes and electrons each migrate toward the oppositely charged electrode. When an electron and hole localize on the same molecule, an “exciton,” which is a localized electron-hole pair having an excited energy state, is formed. Light is emitted when the exciton relaxes via a photoemissive mechanism. In some cases, the exciton may be localized on an excimer or an exciplex. Non-radiative mechanisms, such as thermal relaxation, may also occur, but are generally considered undesirable.

The initial OLEDs used emissive molecules that emitted light from their singlet states (“fluorescence”) as disclosed, for example, in U.S. Pat. No. 4,769,292, which is incorporated by reference in its entirety. Fluorescent emission generally occurs in a time frame of less than 10 nanoseconds.

More recently, OLEDs having emissive materials that emit light from triplet states (“phosphorescence”) have been demonstrated. Baldo et al., “Highly Efficient Phosphorescent Emission from Organic Electroluminescent Devices,” Nature, vol. 395, 151-154, 1998; (“Baldo-I”) and Baldo et al., “Very high-efficiency green organic light-emitting devices based on electrophosphorescence,” Appl. Phys. Lett., vol. 75, No. 3, 4-6 (1999) (“Baldo-II”), which are incorporated by reference in their entireties. Phosphorescence may be referred to as a “forbidden” transition because the transition requires a change in spin states, and quantum mechanics indicates that such a transition is not favored. As a result, phosphorescence generally occurs in a time frame exceeding at least 10 nanoseconds, and typically greater than 100 nanoseconds. If the natural radiative lifetime of phosphorescence is too long, triplets may decay by a non-radiative mechanism, such that no light is emitted. Organic phosphorescence is also often observed in molecules containing heteroatoms with unshared pairs of electrons at very low temperatures. 2,2′-bipyridine is such a molecule. Non-radiative decay mechanisms are typically temperature dependent, such that an organic material that exhibits phosphorescence at liquid nitrogen temperatures typically does not exhibit phosphorescence at room temperature. But, as demonstrated by Baldo, this problem may be addressed by selecting phosphorescent compounds that do phosphoresce at room temperature. Representative emissive layers include doped or un-doped phosphorescent organometallic materials such as disclosed in U.S. Pat. Nos. 6,303,238 and 6,310,360; U.S. Patent Application Publication Nos. 2002-0034656; 2002-0182441; 2003-0072964; and WO-02/074015.

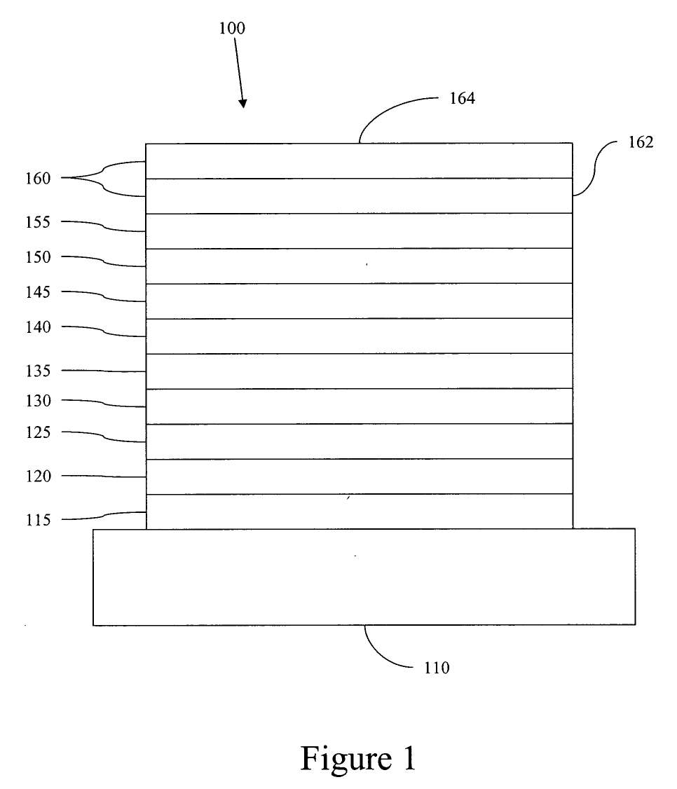

FIG. 1 shows an organic light emitting device 100. The figures are not necessarily drawn to scale. Device 100 may include a substrate 110, an anode 115, a hole injection layer 120, a hole transport layer 125, an electron blocking layer 130, an emissive layer 135, a hole blocking layer 140, an electron transport layer 145, an electron injection layer 150, a protective layer 155, and a cathode 160. Cathode 160 is a compound cathode having a first conductive layer 162 and a second conductive layer 164. Device 100 may be fabricated by depositing the layers described, in order.

Substrate 110 may be any suitable substrate that provides desired structural properties. Substrate 110 may be flexible or rigid. Substrate 110 may be transparent, translucent or opaque. Plastic and glass are examples of preferred rigid substrate materials. Plastic and metal foils are examples of preferred flexible substrate materials. Substrate 110 may be a semiconductor material in order to facilitate the fabrication of circuitry. For example, substrate 110 may be a silicon wafer upon which circuits are fabricated, capable of controlling OLEDs subsequently deposited on the substrate. Other substrates may be used. The material and thickness of substrate 110 may be chosen to obtain desired structural and optical properties.

Anode 115 may be any suitable anode that is sufficiently conductive to transport holes to the organic layers. The material of anode 115 preferably has a work function higher than about 4 eV (a “high work function material”). Preferred anode materials include conductive metal oxides, such as indium tin oxide (ITO) and indium zinc oxide (IZO), aluminum zinc oxide (AlZnO), and metals. Anode 115 (and substrate 110) may be sufficiently transparent to create a bottom-emitting device. A preferred transparent substrate and anode combination is commercially available ITO (anode) deposited on glass or plastic (substrate). A flexible and transparent substrate-anode combination is disclosed in U.S. Pat. Nos. 5,844,363 and 6,602,540 B2, which are incorporated by reference in their entireties. Anode 115 may be opaque and/or reflective. A reflective anode 115 may be preferred for some top-emitting devices, to increase the amount of light emitted from the top of the device. The material and thickness of anode 115 may be chosen to obtain desired conductive and optical properties. Where anode 115 is transparent, there may be a range of thickness for a particular material that is thick enough to provide the desired conductivity, yet thin enough to provide the desired degree of transparency. Other anode materials and structures may be used.

Hole transport layer 125 may include a material capable of transporting holes. Hole transport layer 130 may be intrinsic (undoped), or doped. Doping may be used to enhance conductivity. α-NPD and TPD are examples of intrinsic hole transport layers. An example of a p-doped hole transport layer is m-MTDATA doped with F₄-TCNQ at a molar ratio of 50:1, as disclosed in United States Patent Application Publication No. 2003-02309890 to Forrest et al., which is incorporated by reference in its entirety. Other hole transport layers may be used.

Emissive layer 135 may include an organic material capable of emitting light when a current is passed between anode 115 and cathode 160. Emissive layer 135 contains a fluorescent or phosphorescent emissive material. Emissive layer 135 may also comprise a host material capable of transporting electrons and/or holes, doped with an emissive material that may trap electrons, holes, and/or excitons, such that excitons relax from the emissive material via a photoemissive mechanism. Emissive layer 135 may comprise a single material that combines transport and emissive properties. Whether the emissive material is a dopant or a major constituent, emissive layer 135 may comprise other materials, such as dopants that tune the emission of the emissive material. Emissive layer 135 may include a plurality of emissive materials capable of, in combination, emitting a desired spectrum of light. Examples of fluorescent emissive materials include DCM and DMQA. Examples of phosphorescent emissive materials include Ir(ppy)₃. Examples of host materials include Alq₃, CBP and mCP. Examples of emissive and host materials are disclosed in U.S. Pat. No. 6,303,238 to Thompson et al., which is incorporated by reference in its entirety. Emissive material may be included in emissive layer 135 in a number of ways. For example, an emissive small molecule may be incorporated into a polymer. This may be accomplished by several ways: by doping the small molecule into the polymer either as a separate and distinct molecular species; or by incorporating the small molecule into the backbone of the polymer, so as to form a co-polymer; or by bonding the small molecule as a pendant group on the polymer. Other emissive layer materials and structures may be used. For example, a small molecule emissive material may be present as the core of a dendrimer.

Electron transport layer 145 may include a material capable of transporting electrons. Electron transport layer 145 may be intrinsic (undoped), or doped. Doping may be used to enhance conductivity. Alq₃ is an example of an intrinsic electron transport layer. An example of an n-doped electron transport layer is BPhen doped with Li at a molar ratio of 1:1, as disclosed in United States Patent Application Publication No. 2003-02309890 to Forrest et al., which is incorporated by reference in its entirety. Other electron transport layers may be used.

The charge carrying component of the electron transport layer may be selected such that electrons can be efficiently injected from the cathode into the LUMO (Lowest Unoccupied Molecular Orbital) energy level of the electron transport layer. The “charge carrying component” is the material responsible for the LUMO energy level that actually transports electrons. This component may be the base material, or it may be a dopant. The LUMO energy level of an organic material may be generally characterized by the electron affinity of that material and the relative electron injection efficiency of a cathode may be generally characterized in terms of the work function of the cathode material. This means that the preferred properties of an electron transport layer and the adjacent cathode may be specified in terms of the electron affinity of the charge carrying component of the ETL and the work function of the cathode material. In particular, so as to achieve high electron injection efficiency, the work function of the cathode material is preferably not greater than the electron affinity of the charge carrying component of the electron transport layer by more than about 0.75 eV, more preferably, by not more than about 0.5 eV. Similar considerations apply to any layer into which electrons are being injected.

Cathode 160 may be any suitable material or combination of materials known to the art, such that cathode 160 is capable of conducting electrons and injecting them into the organic layers of device 100. Cathode 160 may be transparent or opaque, and may be reflective. Metals and metal oxides are examples of suitable cathode materials. Cathode 160 may be a single layer, or may have a compound structure. FIG. 1 shows a compound cathode 160 having a thin metal layer 162 and a thicker conductive metal oxide layer 164. In a compound cathode, preferred materials for the thicker layer 164 include ITO, IZO, and other materials known to the art. U.S. Pat. Nos. 5,703,436, 5,707,745, 6,548,956 B2 and 6,576,134 B2, which are incorporated by reference in their entireties, disclose examples of cathodes including compound cathodes having a thin layer of metal such as Mg:Ag with an overlying transparent, electrically-conductive, sputter-deposited ITO layer. The part of cathode 160 that is in contact with the underlying organic layer, whether it is a single layer cathode 160, the thin metal layer 162 of a compound cathode, or some other part, is preferably made of a material having a work function lower than about 4 eV (a “low work function material”). Other cathode materials and structures may be used.

Blocking layers may be used to reduce the number of charge carriers (electrons or holes) and/or excitons that leave the emissive layer. An electron blocking layer 130 may be disposed between emissive layer 135 and the hole transport layer 125, to block electrons from leaving emissive layer 135 in the direction of hole transport layer 125. Similarly, a hole blocking layer 140 may be disposed between emissive layer 135 and electron transport layer 145, to block holes from leaving emissive layer 135 in the direction of electron transport layer 145. Blocking layers may also be used to block excitons from diffusing out of the emissive layer. The theory and use of blocking layers is described in more detail in U.S. Pat. No. 6,097,147 and United States Patent Application Publication No. 2003-02309890 to Forrest et al., which are incorporated by reference in their entireties.

As used herein, and as would be understood by one skilled in the art, the term “blocking layer” means that the layer provides a barrier that significantly inhibits transport of charge carriers and/or excitons through the device, without suggesting that the layer necessarily completely blocks the charge carriers and/or excitons. The presence of such a blocking layer in a device may result in substantially higher efficiencies as compared to a similar device lacking a blocking layer. Also, a blocking layer may be used to confine emission to a desired region of an OLED.

Generally, injection layers are comprised of a material that may improve the injection of charge carriers from one layer, such as an electrode or an organic layer, into an adjacent organic layer. Injection layers may also perform a charge transport function. In device 100, hole injection layer 120 may be any layer that improves the injection of holes from anode 115 into hole transport layer 125. CuPc is an example of a material that may be used as a hole injection layer from an ITO anode 115, and other anodes. In device 100, electron injection layer 150 may be any layer that improves the injection of electrons into electron transport layer 145. LiF/Al is an example of a material that may be used as an electron injection layer into an electron transport layer from an adjacent layer. Other materials or combinations of materials may be used for injection layers. Depending upon the configuration of a particular device, injection layers may be disposed at locations different than those shown in device 100. More examples of injection layers are provided in U.S. patent application Ser. No. 09/931,948 to Lu et al., which is incorporated by reference in its entirety. A hole injection layer may comprise a solution deposited material, such as a spin-coated polymer, erg., PEDOT:PSS, or it may be a vapor deposited small molecule material, e.g., CuPc or MTDATA.

A hole injection layer (HIL) may planarize or wet the anode surface so as to provide efficient hole injection from the anode into the hole injecting material. A hole injection layer may also have a charge carrying component having HOMO (Highest Occupied Molecular Orbital) energy levels that favorably match up, as defined by their herein-described relative ionization potential (IP) energies, with the adjacent anode layer on one side of the HIL and the hole transporting layer on the opposite side of the HIL. The “charge carrying component” is the material responsible for the HOMO energy level that actually transports holes. This component may be the base material of the HIL, or it may be a dopant. Using a doped HIL allows the dopant to be selected for its electrical properties, and the host to be selected for morphological properties such as wetting, flexibility, toughness, etc. Preferred properties for the HIL material are such that holes can be efficiently injected from the anode into the HIL material. In particular, the charge carrying component of the HIL preferably has an IP not more than about 0.7 eV greater that the IP of the anode material. More preferably, the charge carrying component has an IP not more than about 0.5 eV greater than the anode material. Similar considerations apply to any layer into which holes are being injected. HIL materials are further distinguished from conventional hole transporting materials that are typically used in the hole transporting layer of an OLED in that such HIL materials may have a hole conductivity that is substantially less than the hole conductivity of conventional hole transporting materials. The thickness of the HIL of the present invention may be thick enough to help planarize or wet the surface of the anode layer. For example, an HIL thickness of as little as 10 nm may be acceptable for a very smooth anode surface. However, since anode surfaces tend to be very rough, a thickness for the HIL of up to 50 nm may be desired in some cases.

A protective layer may be used to protect underlying layers during subsequent fabrication processes. For example, the processes used to fabricate metal or metal oxide top electrodes may damage organic layers, and a protective layer may be used to reduce or eliminate such damage. In device 100, protective layer 155 may reduce damage to underlying organic layers during the fabrication of cathode 160. Preferably, a protective layer has a high carrier mobility for the type of carrier that it transports (electrons in device 100), such that it does not significantly increase the operating voltage of device 100. CuPc, BCP, and various metal phthalocyanines are examples of materials that may be used in protective layers. Other materials or combinations of materials may be used. The thickness of protective layer 155 is preferably thick enough that there is little or no damage to underlying layers due to fabrication processes that occur after organic protective layer 160 is deposited, yet not so thick as to significantly increase the operating voltage of device 100. Protective layer 155 may be doped to increase its conductivity. For example, a CuPc or BCP protective layer 160 may be doped with Li. A more detailed description of protective layers may be found in U.S. patent application Ser. No. 09/931,948 to Lu et al., which is incorporated by reference in its entirety.

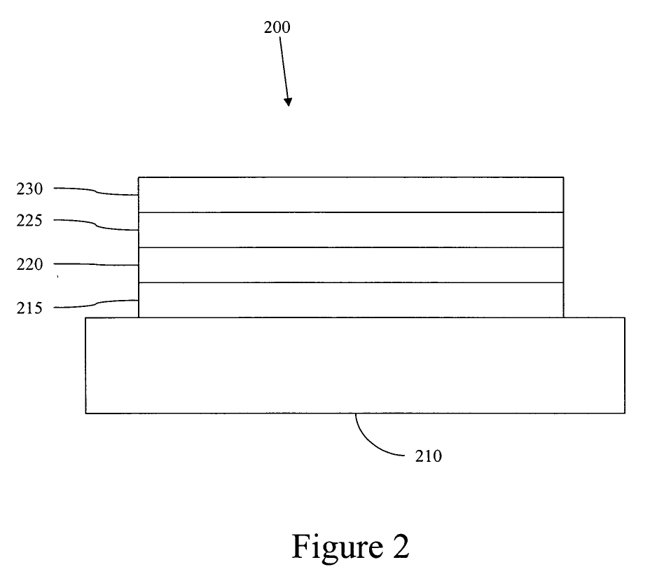

FIG. 2 shows an inverted OLED 200. The device includes a substrate 210, an cathode 215, an emissive layer 220, a hole transport layer 225, and an anode 230. Device 200 may be fabricated by depositing the layers described, in order. Because the most common OLED configuration has a cathode disposed over the anode, and device 200 has cathode 215 disposed under anode 230, device 200 may be referred to as an “inverted” OLED. Materials similar to those described with respect to device 100 may be used in the corresponding layers of device 200. FIG. 2 provides one example of how some layers may be omitted from the structure of device 100.

The simple layered structure illustrated in FIGS. 1 and 2 is provided by way of non-limiting example, and it is understood that embodiments of the invention may be used in connection with a wide variety of other structures. The specific materials and structures described are exemplary in nature, and other materials and structures may be used. Functional OLEDs may be achieved by combining the various layers described in different ways, or layers may be omitted entirely, based on design, performance, and cost factors. Other layers not specifically described may also be included. Materials other than those specifically described may be used. Although many of the examples provided herein describe various layers as comprising a single material, it is understood that combinations of materials, such as a mixture of host and dopant, or more generally a mixture, may be used. Also, the layers may have various sublayers. The names given to the various layers herein are not intended to be strictly limiting. For example, in device 200, hole transport layer 225 transports holes and injects holes into emissive layer 220, and may be described as a hole transport layer or a hole injection layer. In one embodiment, an OLED may be described as having an “organic layer” disposed between a cathode and an anode. This organic layer may comprise a single layer, or may further comprise multiple layers of different organic materials as described, for example, with respect to FIGS. 1 and 2.

Structures and materials not specifically described may also be used, such as OLEDs comprised of polymeric materials (PLEDs) such as disclosed in U.S. Pat. No. 5,247,190, Friend et al., which is incorporated by reference in its entirety. By way of further example, OLEDs having a single organic layer may be used. OLEDs may be stacked, for example as described in U.S. Pat. No. 5,707,745 to Forrest et al, which is incorporated by reference in its entirety. The OLED structure may deviate from the simple layered structure illustrated in FIGS. 1 and 2. For example, the substrate may include an angled reflective surface to improve out-coupling, such as a mesa structure as described in U.S. Pat. No. 6,091,195 to Forrest et al., and/or a pit structure as described in U.S. Pat. No. 5,834,893 to Bulovic et al., which are incorporated by reference in their entireties.

Unless otherwise specified, any of the layers of the various embodiments may be deposited by any suitable method. For the organic layers, preferred methods include thermal evaporation, ink-jet, such as described in U.S. Pat. Nos. 6,013,982 and 6,087,196, which are incorporated by reference in their entireties, organic vapor phase deposition (OVPD), such as described in U.S. Pat. No. 6,337,102 to Forrest et al., which is incorporated by reference in its entirety, and deposition by organic vapor jet printing (OVJP), such as described in U.S. Patent application Ser. No. 10/233,470, which is incorporated by reference in its entirety. Other suitable deposition methods include spin coating and other solution based processes. Solution based processes are preferably carried out in nitrogen or an inert atmosphere. For the other layers, preferred methods include thermal evaporation. Preferred patterning methods include deposition through a mask, cold welding such as described in U.S. Pat. Nos. 6,294,398 and 6,468,819, which are incorporated by reference in their entireties, and patterning associated with some of the deposition methods such as ink-jet and OVJD. Other methods may also be used. The materials to be deposited may be modified to make them compatible with a particular deposition method. For example, substituents such as alkyl and aryl groups, branched or unbranched, and preferably containing at least 3 carbons, may be used in small molecules to enhance their ability to undergo solution processing. Substituents having 20 carbons or more may be used, and 3-20 carbons is a preferred range. Materials with asymmetric structures may have better solution processibility than those having symmetric structures, because asymmetric materials may have a lower tendency to recrystallize. Dendrimer substituents may be used to enhance the ability of small molecules to undergo solution processing.

Devices fabricated in accordance with embodiments of the invention may be incorporated into a wide variety of consumer products, including flat panel displays, computer monitors, televisions, billboards, lights for interior or exterior illumination and/or signaling, heads up displays, fully transparent displays, flexible displays, laser printers, telephones, cell phones, personal digital assistants (PDAs), laptop computers, digital cameras, camcorders, viewfinders, micro-displays, vehicles, a large area wall, theater or stadium screen, or a sign. Various control mechanisms may be used to control devices fabricated in accordance with the present invention, including passive matrix and active matrix. Many of the devices are intended for use in a temperature range comfortable to humans, such as 18 degrees C. to 30 degrees C., and more preferably at room temperature (20-25 degrees C.).

The materials and structures described herein may have applications in devices other than OLEDs. For example, other optoelectronic devices such as organic solar cells and organic photodetectors may employ the materials and structures. More generally, organic devices, such as organic transistors, may employ the materials and structures.

In one embodiment, the present invention provides organic light emitting devices comprising an anode, a cathode, and an organic layer disposed between the anode and the cathode, wherein the organic layer comprises an arylpyrene compound of formula I: 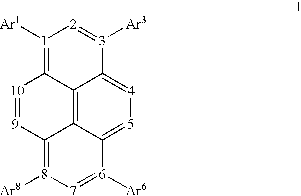
wherein each of Ar¹, Ar³, Ar⁶, and Ar⁸ is independently a 2-napthyl group of structure II: 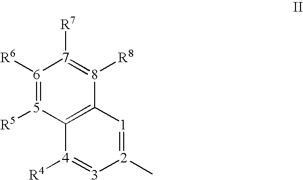
wherein each of R⁴⁻⁸ is an independently selected substituent, and wherein each of Ar¹, Ar³, Ar⁶, and Ar⁸ has a hydrogen at positions 1 and 3. In one embodiment, the present invention provides these compounds per se.

Each of R⁴⁻⁸ is a independently selected substituent. Substituents include, for example, R′, O—R′, N(R′)₂, SR′, C(O)R′, C(O)OR′, C(O)NR′₂, CN, CF₃, NO₂, SO₂, SOR′, SO₃R′, or halo. Each R′ is independently hydrogen, alkyl, alkenyl, alkynyl, heteroalkyl, aralkyl, aryl, or heteroaryl. Any two substituents on adjacent ring atoms can optionally form a 5- or 6-member cyclic group, such that the Ar group is a fused ring group, such as a phenanthryl group. The fused ring may be optionally substituted with one or more substituents, as defined above. In one embodiment, an R group is a straight, branched, or cyclic C₁₋₁₀ alkyl group or a fused ring group. It is believed that alkyl substituents improve the solubility of the compound, thereby making the compound better suited for deposition by solution process. Branched alkyl substituents may be particularly effective at increasing solubility.

Each of Ar¹, Ar³, Ar⁶, and Ar⁸ is independently a 2-napthyl group of structure II, such that the R groups on each of Ar¹, Ar³, Ar⁶, and Ar⁸ may be the same or they may be different. For example, each of R⁴⁻⁸ on Ar¹, Ar³, and Ar⁸ may be a hydrogen, while one of the R groups on Ar⁶ may be, for example, a methyl group:

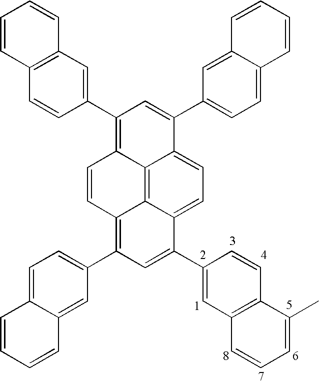

### 1-[2-(5-methylnaphthyl)]-3,6,8-tri(2-naphthyl)pyrene

In one embodiment, each of R⁴⁻⁸ on each of Ar¹, Ar³, Ar⁶, and Ar⁸ is a hydrogen. This unsubstituted form is depicted by formula III:

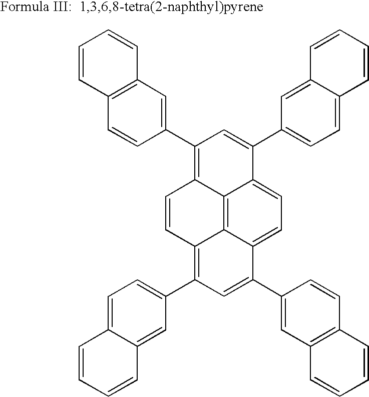

In compounds of embodiments of the present invention, each of Ar¹, Ar³, Ar⁶, and Ar⁸ has a hydrogen at positions 1 and 3. It is believed that this selection increases electron delocalization across the molecule because the Ar groups do not twist relative to the pyrene core, thus increasing π-conjugation.

The π-conjugation in aromatic systems may be increased by extending the π-conjugation by fusing aryl rings or extending the double/triple bonds by, e.g., ortho or para substitutions. It is believed that the oxidized (cation radical) and reduced (anion radical) states of organic materials with high degree of π-conjugation have higher stability than the less conjugated ones. This may be because in the charged state, the hole or electron can delocalize more extensively. Therefore, electron delocalization is desirable because it is believed that such delocalization improves device lifetime. Delocalization may be achieved by increasing conjugation across the molecule. An angle near 90 degrees between the plane of the aryl group and the plane of the pyrene core minimizes conjugation, while a significantly smaller angle may increase conjugation. For example, when one of the 1-, 3-, 6-, and 8-aryl groups is a 1-naphthyl group, the plane of the 1-naphthyl group forms an angle of about 74 degrees with the plane of the pyrene core. On the other hand, when one of the 1-, 3-, 6-, and 8-aryl groups is a 2-naphthyl group, the plane of the 2-naphthyl group forms a substantially smaller angle of about 51 degrees with the plane of the pyrene core. This small dihedral angle between 2 aromatic rings provides more π-conjugation. Thus, the 2-naphthyl analog provides more π-conjugation than the 1-naphthyl analog. In one embodiment, the arylpyrene compound of the present invention has a dihedral angle of less than about 60 degrees. The dihedral angle can be obtained from the optimized equilibrium geometry after energy minimization followed by density function theory (DFT) calculation (B3LYP 6-31 G*) using the Spartan 02 software package.

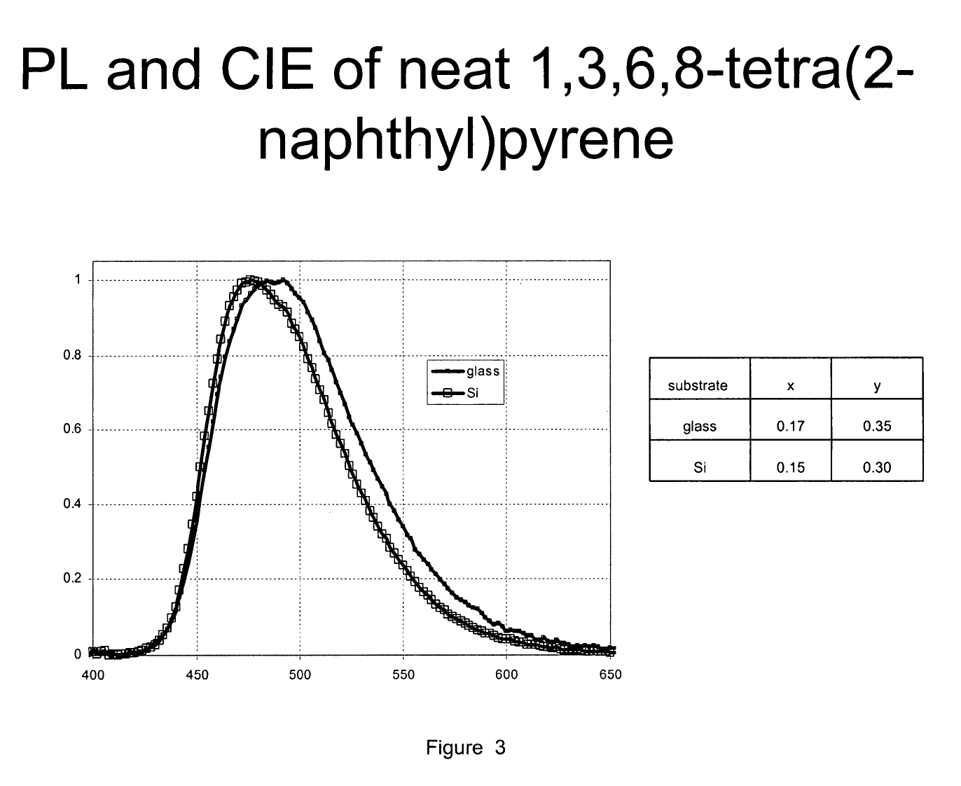

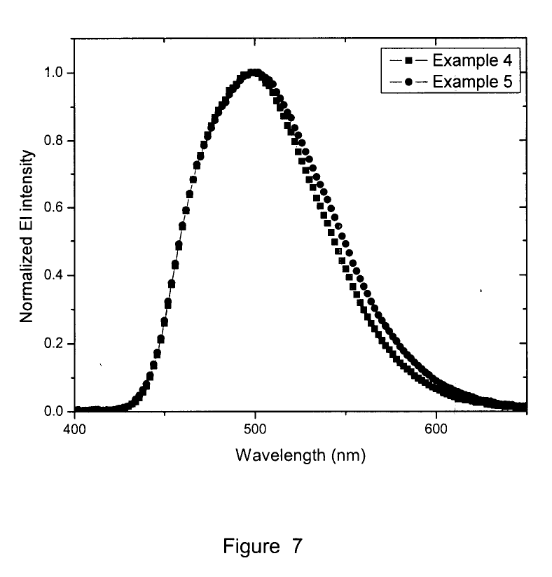

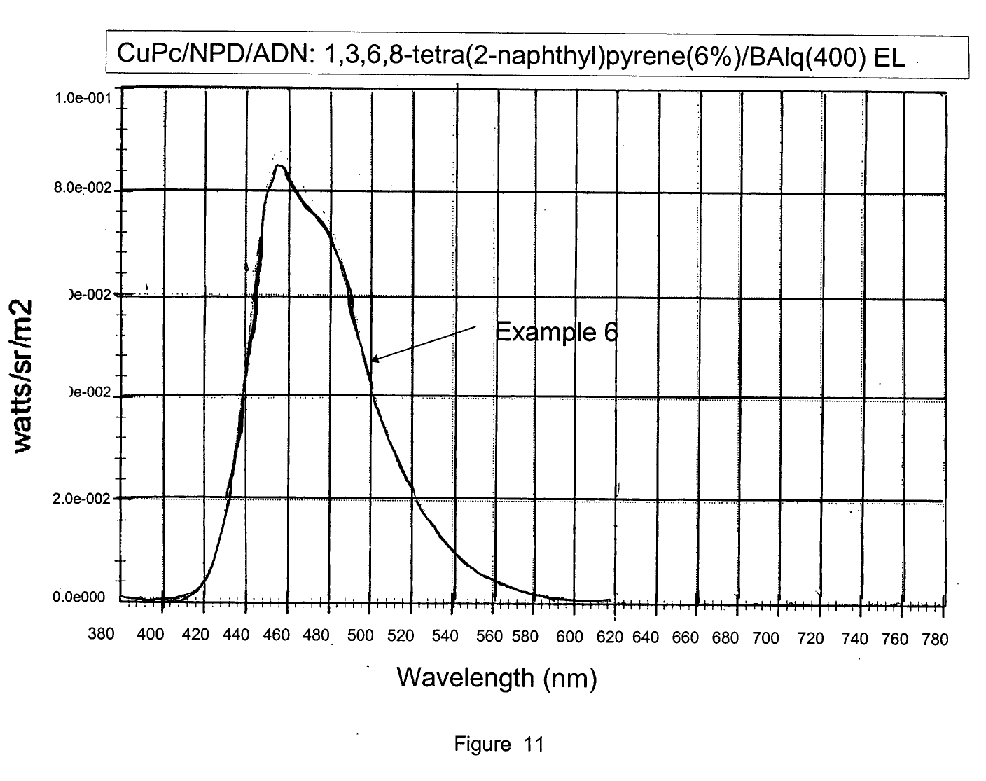

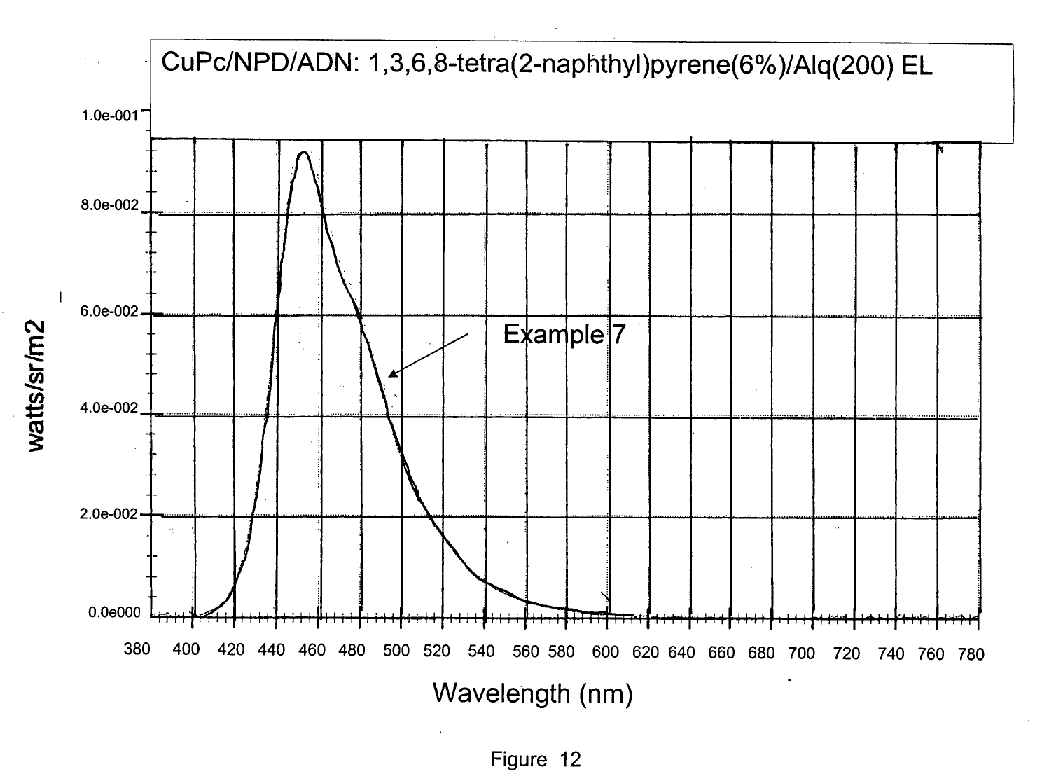

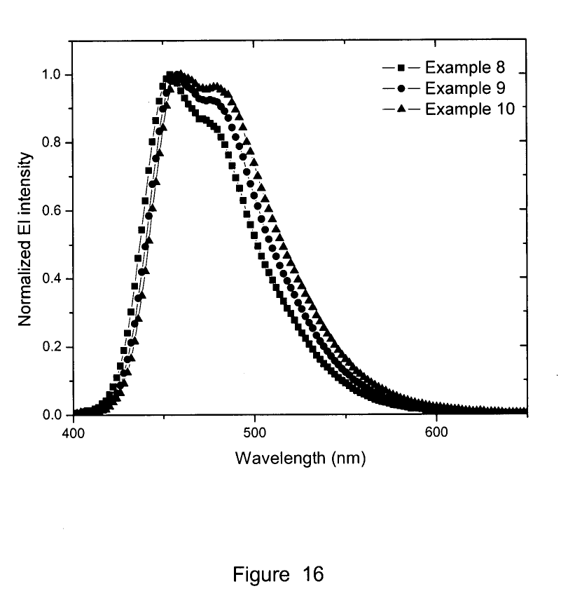

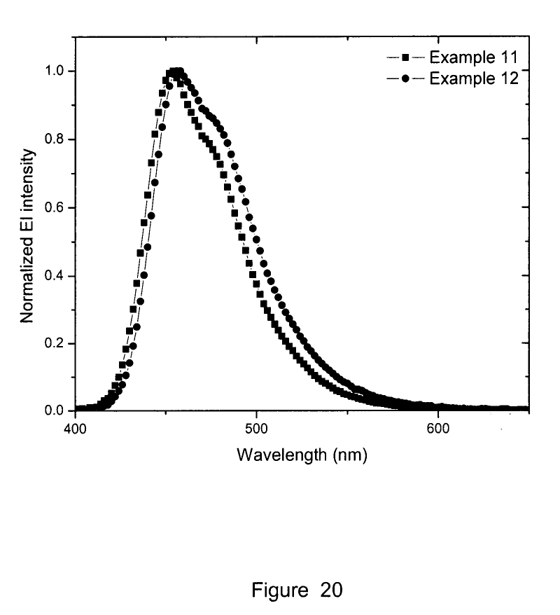

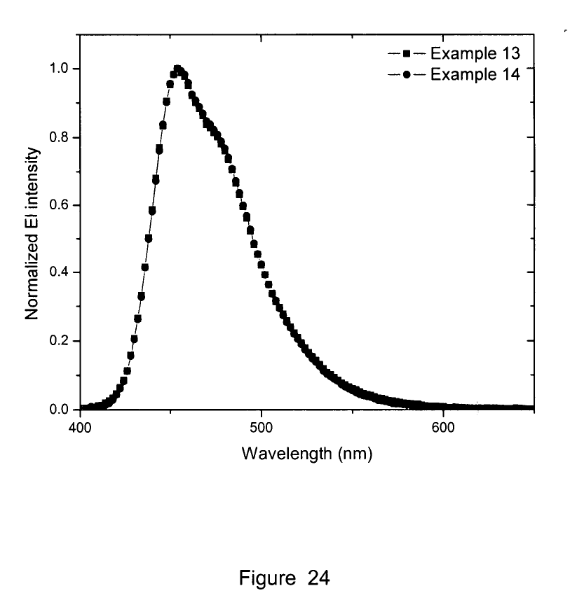

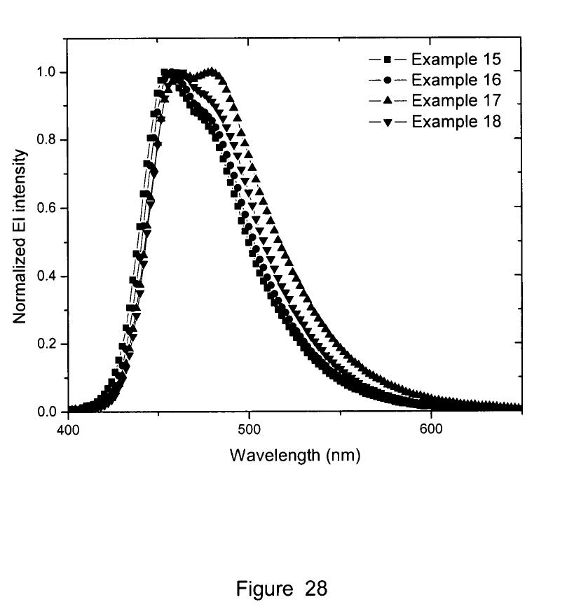

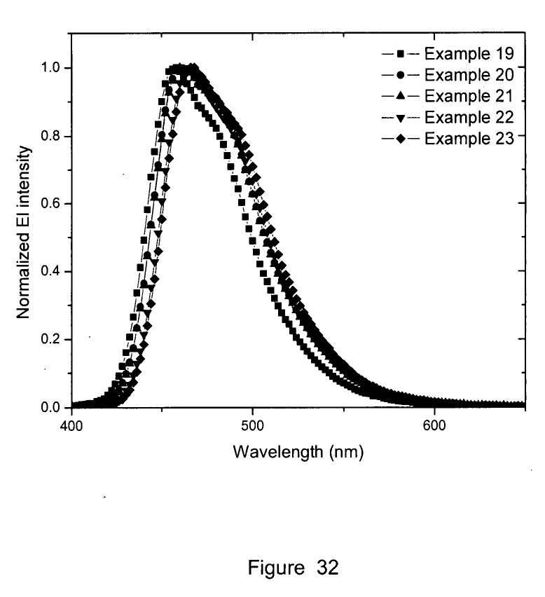

In one embodiment, the present invention provides a device comprising an arylpyrene compound that emits blue light, particularly light with CIE coordinates of (X≦0.2, Y≦0.3). In a preferred embodiment, the compound has a peak in the emission spectra that is less than about 500 nm, preferably less than 450 nm. See, e.g., FIGS. 3, 7, 11, 12, 16, 20, 24, 28, and 32. Arylpyrene compounds, particularly 1,3,6,8-tetraarylpyrenes, are useful as fluorescent emitters capable of emitting blue light. However, pyrene compounds in solid state are susceptible to pyrene π-stacking, which causes red-shifting of the emission. Tuning the emission color can be accomplished by using substituents to control the degree of π-stacking. For example, adding branched alkyl substituents onto the aryl groups may inhibit π-stacking. The degree of π-stacking is also influenced by the concentration of the compound in solution. Diluting the compound reduces π-stacking.

Comparative examples of compounds of formula I include those in which the Ar group is a fused ring group selected from the group consisting of 2-naphthyl, 9-phenanthryl, and 9-anthryl. One or more of Ar¹, Ar³, Ar⁶, and Ar⁸ can be independently substituted with one or more substituents. The position of substituents is described according to the following numbering:

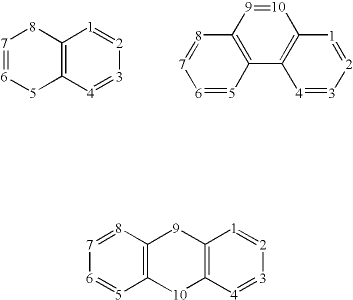

For example, the comparative example in which Ar is 9-phenanthryl is demonstrated in its unsubstituted form by formula IV:

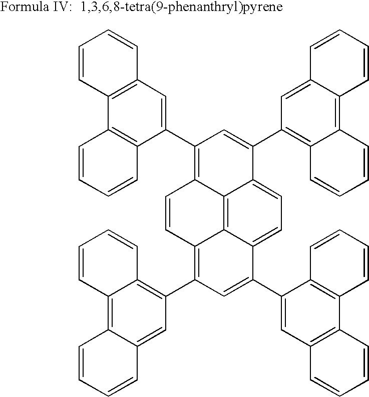

The comparative example in which Ar is 9-anthryl is demonstrated in its unsubstituted form by formula V:

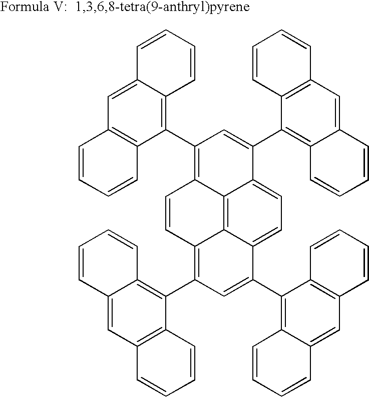

In one embodiment, the arylpyrene compound and/or other layers of the device are deposited as a neat layer. Blue emission from a neat layer of an arylpyrene compound is possible by controlling the degree of π-stacking, as described above. As appreciated by one of skill in the art, neat layers offer advantageous ease of device fabrication and reduced voltage rise. By avoiding the use of a host, which generally has a wider band gap than the dopant, stability may be increased. As a result, not as much π-conjugation may be needed to achieve a desirable stability.

Alternatively, the arylpyrene compound and/or other layers of the device may be fabricated by organic vapor phase deposition (OVPD) or by ink-jet printing.

In one embodiment, the arylpyrene compound is doped in a host material. The host material can be, for example, an anthracene host, such as ADN, or a carbazole host, such as CBP. Preferably, the host material is an anthracene host material, depicted by the general formula VI:

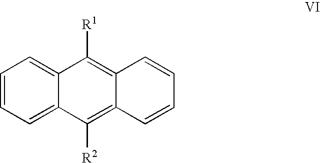

Each of R¹ and R² is independently an optionally substituted aromatic group. Anthracene hosts include, but are not limited to 9,10-di(2-naphthyl)anthracene (ADN) and 9,10-di[3-(N-carbazole)phenyl]anthracene (B3CPA). In a preferred embodiment, the anthracene host is ADN. In another preferred embodiment, the anthracene host is ADN, and the arylpyrene compound is 1,3,6,8-tetra(2-naphthyl)pyrene (III).

In one embodiment, the devices of the present invention have an unmodified external quantum efficiency greater than about 5%. The term “unmodified external quantum efficiency” as used herein refers to the external quantum efficiency of a device, after multiplication by a factor to account for any differences in the out-coupling efficiency of that device and the out-coupling efficiency of the devices described experimentally herein. For example, a device having an external quantum efficiency of 5%, but having an out-coupling efficiency 3 times better than the devices described herein, would have an “unmodified external quantum efficiency” of 1.33% (one third of 5%). A typical out-coupling efficiency for the types of devices described herein is about 20-30%. Out-coupling may be improved, for example, by incorporating features such as a mesa structure as described in U.S. Pat. No. 6,091,195 to Forrest et al., and/or a pit structure as described in U.S. Pat. No. 5,834,893 to Bulovic et al. Other methods of improving out-coupling may also be used. There are device structures having better out-coupling efficiencies than the devices described herein, and it is anticipated that improvements to out-coupling efficiency will be made over time. Such improvements would enhance external quantum efficiency, but should not affect “unmodified” external quantum efficiency, and devices having such improvements may fall within the scope of the present invention.

It is understood that the various embodiments described herein are by way of example only, and are not intended to limit the scope of the invention. For example, many of the materials and structures described herein may be substituted with other materials and structures without deviating from the spirit of the invention. It is understood that various theories as to why the invention works are not intended to be limiting. For example, theories relating to charge transfer are not intended to be limiting.

Material Definitions:

As used herein, abbreviations refer to materials as follows:
α-NPD: N,N′-diphenyl-N—N′-di(1-naphthyl)-benzidine
ADN: 9,10-di(2-naphthyl)anthracene
CBP: 4,4′-N,N-dicarbazolebiphenyl
m-MTDATA 4,4′,4″-tris(3-methylphenylphenlyamino)triphenylamine
Alq₃: tris(8-hydroxyquinoline) aluminum
Bphen: 4,7-diphenyl-1,10-phenanthroline
n-BPhen: n-doped BPhen (doped with lithium)
F₄-TCNQ: tetrafluoro-tetracyano-quinodimethane
p-MTDATA: p-doped m-MTDATA (doped with F₄-TCNQ)
Ir(ppy)₃: tris(2-phenylpyridine)iridium
Ir(ppz)₃: tris(1-phenylpyrazoloto,N,C(2′)iridium(III)
BCP: 2,9-dimethyl-4,7-diphenyl-1,10-phenanthroline
TAZ: 3-phenyl-4-(1′-naphthyl)-5-phenyl-1,2,4-triazole
CuPc: copper phthalocyanine.
ITO: indium tin oxide
NPD: N,N′-diphenyl-N—N′-di(1-naphthyl)-benzidine
TPD: N,N′-diphenyl-N—N′-di(3-toly)-benzidine
BAlq: aluminum(III)bis(2-methyl-8-hydroxyquinolinato)₄-phenylphenolate
mCP: 1,3-N,N-dicarbazole-benzene
DCM: 4-(dicyanoethylene)-6-(4-dimethylaminostyryl-2-methyl)-4H-pyran
DMQA: N,N′-dimethylquinacridone
PEDOT:PSS: an aqueous dispersion of poly(3,4-ethylenedioxythiophene) with polystyrenesulfonate (PSS)
mCBP 3,3′-N,N-dicarbazole-biphenyl
2,7-DCP: 2,7-N,N-dicarbazolephenanthrene
HPT: 2,3,6,7,10,11-hexaphenyltriphenylene
B3CPA 9,10-Di[3-(N-carbazole)phenyl]anthracene

### EXPERIMENTAL

Specific representative embodiments of the invention will now be described, including how such embodiments may be made. It is understood that the specific methods, materials, conditions, process parameters, apparatus and the like do not necessarily limit the scope of the invention.

### Example 1a

### Synthesis of 1,3,6,8-tetra(2-naphthyl)pyrene

Step 1. 1,3,6,8-Tetrabromopyrene

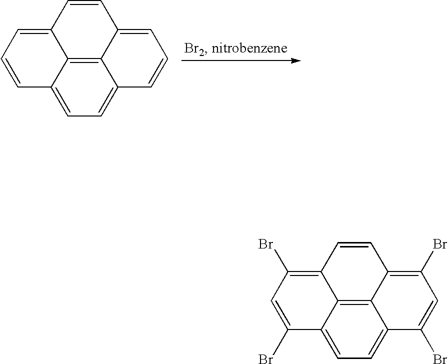

Bromine (39.5 g, 247.2 mmol) was added via an addition funnel to a solution of pyrene (10.0 g, 49.4 mmol) in 250 mL of nitrobenzene at room temperature. While stirring vigorously with a mechanical stirrer, this reaction was heated to 120° C. and left at reflux for 24 hours while ensuring continuous stirring. After cooling, the solid was filtered, washed with ethanol, and dried. The crude yield was about 47%. Recrystallization in 1,2-dichlorobenzene yielded yellow needle-like solids.
Step 2. 1,3,6,8-Tetra(2-naphthyl)pyrene

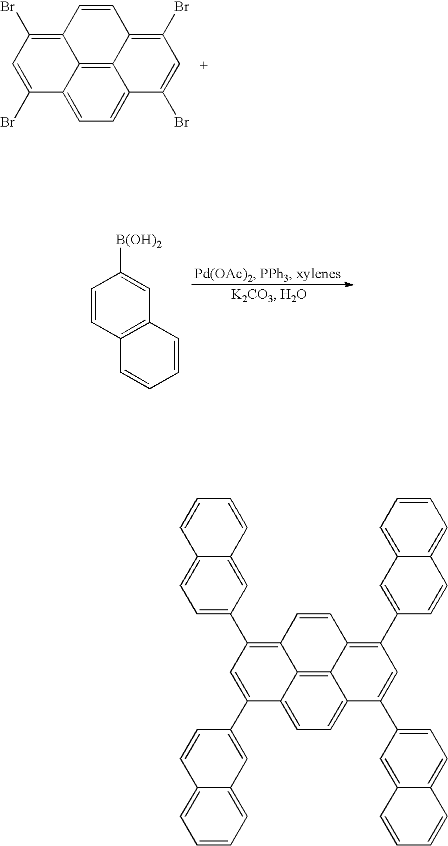

1,3,6,8-Tetrabromopyrene (6.0 g, 11.6 mmol), 2-naphthylboronic acid (10.0 g, 57.9 mmol), triphenylphosphine (1.2 g, 4.6 mmol), palladium(II) acetate (0.3 g, 1.2 mmol), and potassium carbonate (11.2 g, 81.1 mmol) were mixed in 350 mL of xylenes and 70 mL of water. The mixture was purged with nitrogen for ten minutes and slowly brought to reflux under nitrogen. The mixture was refluxed for 41 hours. After cooling, the solid was filtered, washed with water and ethanol, and dried. The dried material (about 6-8 g) was sublimed using the following conditions under vacuum of about 1×10⁻⁵ torr. The sublimed material from was then collected, which resulted in about 5 g of crude product. Recrystallization in xylenes (about 160 mL per 4 g) yielded a yellow solid, which was then sublimed again under a vacuum of about 1×10⁻⁵ torr. The product was characterized by mass spectrometry.

### Example 1b

### Synthesis of 1,3,6,8-tetra(9-phenanthryl)pyrene

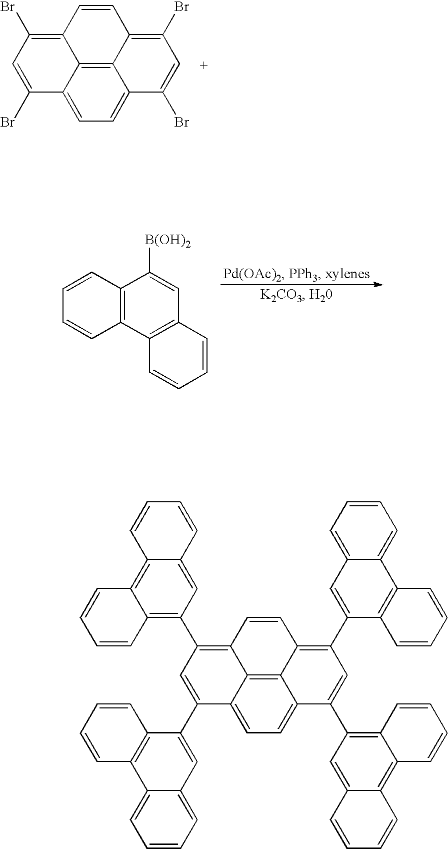

1,3,6,8-tetrabromopyrene (4.7 g, 9.1 mmol), 9-phenanthreneboronic acid (11.7 g, 45.4 mmol), triphenylphosphine (1.0 g, 3.6 mmol), palladium(II) acetate (0.2 g, 0.9 mmol), and potassium carbonate (8.8 g, 63.5 mmol) were in 300 mL of xylenes and 50 mL of water. The mixture was purged with nitrogen for ten minutes and slowly brought to reflux under nitrogen. The mixture was refluxed for forty-eight hours. After cooling, the solid was filtered, washed with water and ethanol, and dried. The crude yield was ˜95%. Based on the purification of 1,3,6,8-tetra(2-naphthyl)pyrene, sublimation was the most effective means of purifying this crude material; however, 1,3,6,8-tetra(9-phenanthryl)pyrene decomposed under similar sublimation conditions as in 1,3,6,8-tetra(2-naphthyl)pyrene.

### Comparative Example 1

### Synthesis of 1,3,6,8-tetra(1-naphthyl)pyrene

1,3,6,8-Tetrabromopyrene and 1-naphthylboronic acid were reacted under the same condition as in the synthesis of 1,3,6,8-tetra(2-naphthyl)pyrene. No isolatable amount of the desired product was formed.

### Example 2

### 9,10-Di(2-naphthyl)anthracene (ADN)

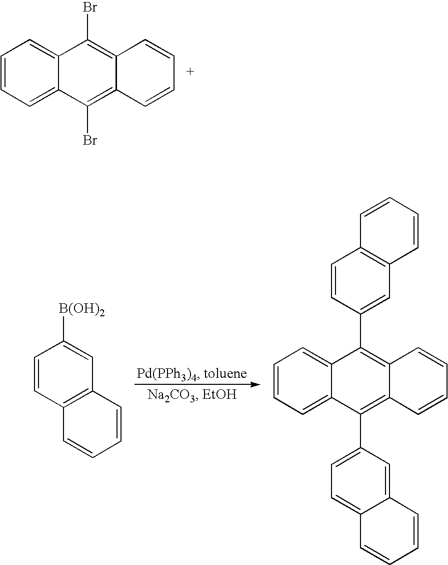

9,10-dibromoanthracene (6.0 g, 17.9 mmol) and 2-naphthylene boronic acid (7.1 g, 41.1 mmol) were added to 100 mL of toluene and 17 mL of ethanol. 50 mL of 2.0 M aqueous Na₂CO₃ was added, and the reaction mixture was purged for ten minutes before adding tetrakis(triphenylphospine)palladium(0) (0.03 g, 0.14 mmol). The reaction was left at reflux for 24 hours while under nitrogen. While hot, the organic suspension layer was separated, added to 50 mL of a 2.0 N HCl solution, and left at reflux for one hour. The organic layer was then washed three times with water until neutral pH was reached and filtered. This filtrate was then washed with a small amount of cold acetone followed by toluene and then dried. The crude yield was ˜62%. Recrystallization in xylenes (˜290 mL per 4.8 g) yielded an off-white crystalline solid. This solid was then sublimed under vacuum. The material was characterized by mass spectrometry.

### Example 3

### Synthesis of 9,10-Di[3-(N-carbazole)phenyl]anthracene

Step 1: Bromo-3-(N-carbazole)benzene

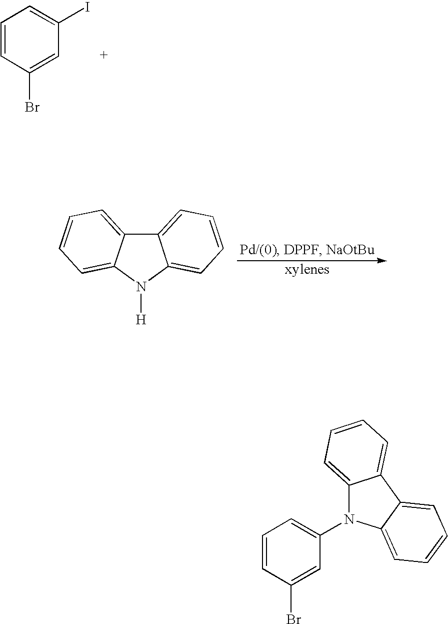

Tris(dibenzylideneacetone)dipalladium(0) (0.4 g, 0.4 mmol), 1,1′-bis(diphenylphosphino)ferrocene (0.4 g, 0.7 mmol), and sodium tert-butoxide (4.3 g, 44.2 mmol) were added to a dry flask under nitrogen before adding 100 mL of xylenes and stirring at room temperature for 15 minutes. 1-bromo-3-iodobenzene (25.0 g, 88.4 mmol) was then added to the reaction mixture and left to stir for another 15 minutes. Carbazole (4.9 g, 29.5 mmol) was then added and the reaction mixture was brought to reflux. The reaction was left at reflux for 24 hours under nitrogen. Once cooled, the reaction mixture was filtered in order to remove the catalyst, and the solids were rinsed with hexanes. The resulting filtrate was rotovaped resulting in a viscous, brown-red liquid which was then column chromatographed with 20/80 methylene chloride/hexanes to yield a viscous yellow-orange liquid. The yield was ˜96%.
Step 2: 3-(N-carbazole)phenylboronic acid

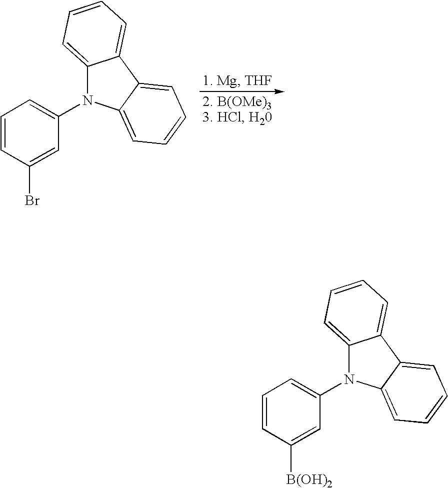

9-(3-bromophenyl)carbazole (8.3 g, 25.8 mmol) was transferred to a dry three-neck flask. Nitrogen was purged through the system for about ten minutes before the addition of 51 mL of dry THF via syringe. Magnesium turnings (1.3 g, 51.5 mmol) was then added to this solution, which was then heated slowly with a heat gun in order to accelerate the formation of the Grignard reagent. The reaction mixture turned color from a light yellow to a dark yellow-brown solution. Once the exothermic reaction had subsided, the solution was cannulated to another dry three-neck flask before cooling the flask to 0° C. Trimethylborate (5.7 mL, 51.5 mmol) was added via syringe, which caused the solution to become a milky white color. After stirring overnight, 30 mL of 2.0 M HCl and 60 mL of methylene chloride were added to the reaction and left to stir for 15 minutes. The organic layer was separated, dried using Na₂SO₄, and rotovaped resulting in a viscous, yellow liquid which was then column chromatographed using 50/50/5% methylene chloride/hexanes/methanol to yield an off-white solid. The yield was ˜73%. The product was characterized mass spectrometry.
Step 3: 9,10-Di [3-(N-carbazole)phenyl]anthracene (B3CPA)

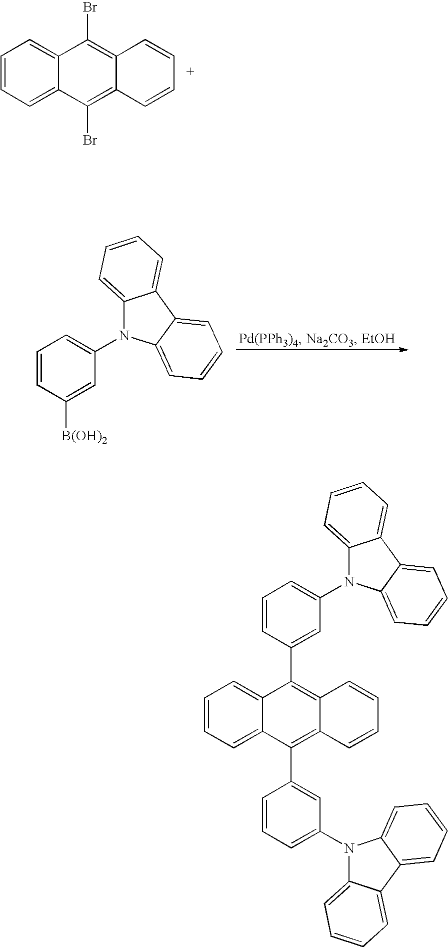

9,10-dibromoanthracene (2.6 g, 7.9 mmol) and 3-(N-carbazole)phenylboronic acid (5.4 g, 18.9 mmol) were added to 100 mL of toluene and 20 mL of ethanol. 50 mL of 2.0 M aqueous Na₂CO₃ was added, and the reaction mixture was purged for ten minutes before adding tetrakis(triphenylphosphine)palladium(0) (0.07 g, 0.06 mmol). The reaction was left at reflux for forty-three hours while under nitrogen. While the reaction mixture was warm, it was filtered, and the solids were washed with water and ethanol. The crude yield was ˜95%. Recrystallization in toluene (˜500 mL per 4.9 g) yielded off-white crystalline solids, which were then purified by vacuum sublimation. The product was characterized by mass spectrometry.

Device Fabrication and Measurement

All devices are fabricated by high vacuum (<10⁻⁷ Torr) thermal evaporation. The anodes are about 800 Å and about 1200 Å thick indium tin oxide (ITO) for examples 6, 7, 11-23 and 4, 5, 8-10 respectively. The cathode consists of 10 Å of LiF followed by 1,000 Å of Al. All devices are encapsulated with a glass lid sealed with an epoxy resin in a nitrogen glove box (<1 ppm of H₂O and O₂) immediately after fabrication, and a moisture getter was incorporated inside the package.

### Examples 4-23

The organic stack consists of sequentially, from the ITO surface, 100 Å of copper phthalocyanine (CuPc) as the hole injection layer (HIL), 300 Å of 4,4′-bis[N-(1-naphthyl)-N-phenylamino]biphenyl (α-NPD), as the hole transporting layer (HTL), 300 Å of a host doped with 3-18 wt % of 1,3,6,8-tetra(2-naphthyl)pyrene or 300 Å of neat 1,3,6,8-tetra(2-naphthyl)pyrene as the emissive layer (EML), 100-400 Å of an ETL2 and 300-400 Å of an ETL1. The device composition (host, doping level, ETL2 and ETL1), CIE coordinates, external quantum efficiency, and luminous efficiency are summarized in the Table 1.

TABLE 1

| | | 1,3,6,8- | | | | | |
|---|---|---|---|---|---|---|---|
| | | tetra(2- | | | | | External |
| | | naphthyl- | | | | Luminous | quantum |
| | | pyrene) | ETL2 | ETL1 | | efficiency | efficiency |
| | | doping | (thickness | (thickness | | (cd/A) at | (%) at 100 |
| Example | Host | level (%) | in Å) | in Å | CIE | 100 cd/m² | cd/m² |
| 4 | none | 100 | BAlq (400) | none | 0.177, 0.407 | 8.8 | 3.6 |
| 5 | none | 100 | HPT (100) | BAlq (400) | 0.191, 0.419 | 8.8 | 3.5 |
| 6 | ADN | 6 | BAlq (400) | none | 0.141, 0.149 | 7.9 | 6.6 |
| 7 | ADN | 6 | Alq₃ (200) | none | 0.144, 0.116 | 6.6 | 6.5 |
| 8 | CBP | 6 | BAlq (400) | none | 0.142, 0.154 | 2.9 | 2.3 |
| 9 | CBP | 12 | BAlq (400) | none | 0.142, 0.185 | 4.9 | 3.5 |
| 10 | CBP | 18 | BAlq (400) | none | 0.145, 0.213 | 5.8 | 3.7 |
| 11 | mCP | 6 | HPT (100) | BAlq (300) | 0.140, 0.115 | 3.4 | 3.4 |
| 12 | mCP | 12 | HPT (100) | BAlq (300) | 0.140, 0.152 | 4.7 | 3.8 |
| 13 | mCBP | 6 | BAlq (400) | none | 0.141, 0.130 | 3.6 | 3.3 |
| 14 | mCBP | 6 | HPT (100) | BAlq (300) | 0.139, 0.128 | 4.7 | 4.4 |
| 15 | B3CPA | 3 | BAlq (400) | none | 0.143, 0.153 | 5.9 | 4.8 |
| 16 | B3CPA | 6 | BAlq (400) | none | 0.142, 0.167 | 6.3 | 4.8 |
| 17 | B3CPA | 6 | BAlq (100) | Alq₃ (400) | 0.151, 0.227 | 6.3 | 3.8 |
| 18 | B3CPA | 12 | BAlq (400) | none | 0.142, 0.193 | 6.9 | 4.8 |
| 19 | 2,7-DCP | 6 | BAlq (400) | none | 0.139, 0.146 | 4.7 | 4 |
| 20 | 2,7-DCP | 12 | BAlq (400) | none | 0.139, 0.183 | 6.4 | 4.6 |
| 21 | 2,7-DCP | 12 | HPT (100) | BAlq (300) | 0.139, 0.184 | 6.8 | 4.9 |
| 22 | HPT | 12 | BAlq (400) | none | 0.137, 0.205 | 7.2 | 4.9 |
| 23 | HPT | 18 | BAlq (400) | none | 0.137, 0.219 | 7.8 | 5.1 |

It can be seen from the device summary table that the invention compound 1,3,6,8-Tetra(2-naphthyl)pyrene generates efficient and deep blue devices with a variety of host materials including carbazole and anthracene hosts. Examples 6 and 7 represent some of the most efficient fluorescent OLEDs in the deep blue color region to date. The stability is also outstanding. Operating under a DC current of 5 mA/cm², the T_{1/2} (defined by the time required to reach 50% of the initial luminescence L₀) are projected to >5000 hrs for Example 6 (L₀=450 cd/m²) and >6000 hrs for Example 7 (L₀=340 cd/m²) respectively. These lifetimes also represent some of the most stable OLEDs in this deep blue color region to date.

While the present invention is described with respect to particular examples and preferred embodiments, it is understood that the present invention is not limited to these examples and embodiments. The present invention as claimed therefore includes variations from the particular examples and preferred embodiments described herein, as will be apparent to one of skill in the art.

What is claimed is:

1. An organic light emitting device comprising:
a) an anode;
b) a cathode; and
c) an organic layer disposed between the anode and the cathode, wherein the organic layer comprises an arylpyrene compound of formula I: 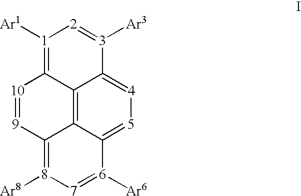
wherein each of Ar¹, Ar³, Ar⁶, and Ar⁸ is independently a 2-napthyl group of structure II: 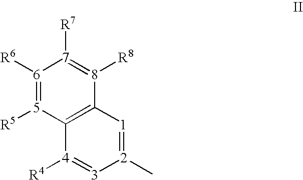
wherein each of R⁴⁻⁸ is an independently selected substituent, and wherein each of Ar¹, Ar³, Ar⁶, and Ar⁸ has a hydrogen at positions 1 and 3.

2. The device of claim 1, wherein the arylpyrene compound is a compound of formula III:

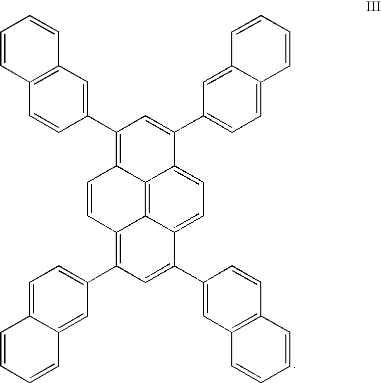

3. The device of claim 1, wherein the arylpyrene compound has an angle defined by the plane of the pyrene core and the plane of the Ar¹, Ar³, Ar⁶, or Ar⁸ group that is less than about 60 degrees.

4. The device of claim 1, wherein the arylpyrene compound is doped in a host.

5. The device of claim 4, wherein the host is an anthracene host.

6. The device of claim 5, wherein the anthracene host is ADN.

7. The device of claim 4, wherein the host is a carbazole host.

8. The device of claim 7, wherein the carbazole host is CBP.

9. The device of claim 1, wherein the arylpyrene compound is deposited as a neat layer.

10. The device of claim 1, wherein the arylpyrene compound has a peak in the emission spectra that is less than about 500 mm.

11. The device of claim 10, wherein the arylpyrene compound emits light with CIE coordinates of (X≦0.2, Y≦0.3).

12. The device of claim 1, wherein the unmodified external quantum efficiency is greater than about 5%.

13. An arylpyrene compound of formula I: 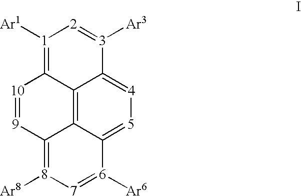
wherein each of Ar¹, Ar³, Ar⁶, and Ar⁸ is independently a 2-napthyl group of structure II: 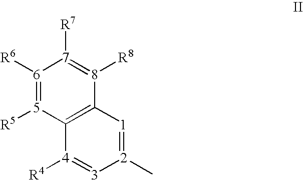
wherein each of R⁴⁻⁸ is an independently selected substituent, and wherein each of Ar¹, Ar³, Ar⁶, and Ar⁸ has a hydrogen at positions 1 and 3.

14. The compound of claim 13, wherein the arylpyrene compound is a compound of formula III: [chemical structure]
